(19) United States
(12) Patent Application Publication
(10) Pub. No.: US 2011/0197346 A1
(43) Pub. Date: Aug. 18, 2011

(54) GOGGLES WITH REPLACEABLE STRAP ATTACHMENTS

(76) Inventor: Roy Burchett, La Verne, CA (US)

(21) Appl. No.: 12/707,965

(22) Filed: Feb. 18, 2010

(51) Int. Cl. A61F 9/02

(52) U.S. Cl. 2/431; 2/452

(57) ABSTRACT

A pair of goggles comprises a frame, a lens, a first goggle outrigger and a second goggle outrigger. Both goggle outriggers include a body and a pair of opposed struts. Each distal end of each goggle outrigger strut has a frame attachment device. Each frame attachment device is attached to a goggle outrigger strut attachment device, such that each goggle outrigger is hingedly affixed to the frame and such that the body of each goggle outrigger can be articulated away from the frame. Each goggle outrigger can be easily deattached from and reattached to the frame without the use of tools.

### BACKGROUND OF THE INVENTION

Goggles, such as those worn by sports participants and motorcycle riders, typically are retained upon the head of the wearer by an elastic strap. Such elastic strap is adapted at each end to attach to respective attachment connections on either side of the goggles.

The attachment connections on either side of the goggles are often of a contrasting color or design with respect to that of the remainder of the goggles. Such contrasting color and/or design is frequently seen as desirable by users of the goggles.

It would be advantageous if such strap attachment connections could be periodically and easily replaced with attachment connections having a different color and/or design.

### SUMMARY OF THE INVENTION

In one aspect of the invention, the invention comprises a pair of goggles comprising (a) a frame defining a central opening, the frame having a middle portion, a first side portion and a second side portion, each side portion having a pair of opposed goggle outrigger strut attachment devices; (b) a lens attached within the central opening of the frame; (c) a first goggle outrigger attached to the first side portion of the frame, the first goggle outrigger comprising a body and a pair of opposed struts, each opposed strut of the first goggle outrigger having a proximal end attached to the body of the first goggle outrigger and a free distal end, each distal end having a frame attachment device; and (d) a second goggle outrigger attached to the second side portion of the frame, the second goggle outrigger comprising a body and a pair of opposed struts, each opposed strut of the second goggle outrigger having a proximal end attached to the body of the second goggle outrigger and a free distal end, each distal end having a frame attachment device. In this aspect of the invention, each frame attachment device on each distal end of each strut of each goggle outrigger is attached to a goggle outrigger strut attachment device on the frame, such that each goggle outrigger is hingedly affixed to the frame, and such that the body of each goggle outrigger can be articulated away from the frame, and each goggle outrigger can be easily deattached from and reattached to the frame without the use of tools.

In another aspect of the invention, the invention comprises (a) a frame defining a central opening, the frame having a middle portion, a first side portion and a second side portion, each side portion having a pair of opposed goggle outrigger strut attachment devices; (b) a lens attached within the central opening of the frame; (c) a first goggle outrigger attached to the first side portion of the frame, the first goggle outrigger comprising a body and a pair of opposed struts, the body of the first goggle outrigger having at least one strap outrigger attachment device, each opposed strut of the first goggle outrigger having a proximal end attached to the body of the first goggle outrigger and a free distal end, each distal end having a frame attachment device; (d) a second goggle outrigger attached to the second side portion of the frame, the second goggle outrigger comprising a body and a pair of opposed struts, the body of the second goggle outrigger having at least one strap outrigger attachment device, each opposed strut of the second goggle outrigger having a proximal end attached to the body of the second goggle outrigger and a free distal end, each distal end having a frame attachment device; (e) a first strap outrigger attached to the body of the first goggle outrigger, the first strap outrigger comprising a body and at least one goggle outrigger body attachment device; and (f) a second strap outrigger attached to the body of the second goggle outrigger, the second strap outrigger comprising a body and at least one goggle outrigger body attachment device. In this aspect of the invention, each frame attachment device on each distal end of each strut of each goggle outrigger is attached to a goggle outrigger strut attachment device on the frame, such that each goggle outrigger is hingedly affixed to the frame, and such that the body of each goggle outrigger can be articulated away from the frame, and each goggle outrigger body attachment device is attached to a strut outrigger attachment device such that each strap outrigger can easily be deattached from and reattached to a goggle outrigger without the use of tools.

### DRAWINGS

These features, aspects and advantages of the present invention will become better understood with regard to the following description, appended claims and accompanying figures where:

FIG. 1 is perspective view of a pair of goggles having features of the invention;

FIG. 2 is a second perspective view of the pair of goggles illustrated in FIG. 1, illustrating the upper portion of those goggles;

FIG. 3 is a perspective view of the goggles illustrated in FIG. 1, illustrating the attachment of a strap to the goggles;

FIG. 4 is a perspective view of the goggles illustrated in FIG. 1, shown without a strap and strap attachment system;

FIG. 5 is a first exploded detail view of a goggle attachment system useable in the invention;

FIG. 6 is a perspective view of a goggle outrigger strut attachment device useable in the invention;

FIG. 7 is a second exploded view of the goggle attachment system illustrated in FIGS. 5; and

FIG. 8 is a perspective view of the goggle attachment system illustrated in FIG. 5, showing the goggle attachment system fully assembled.

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

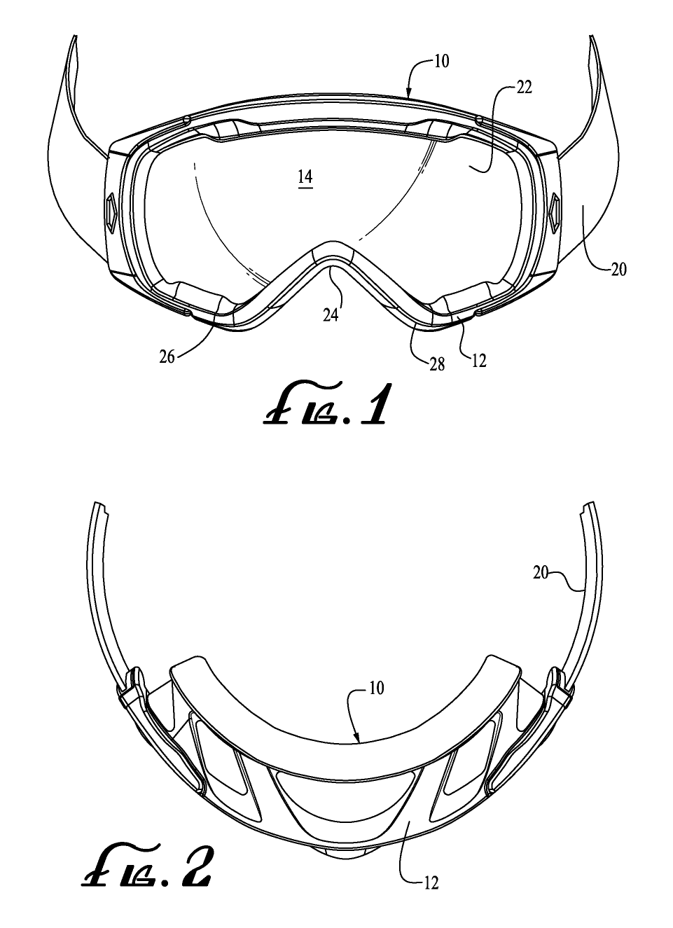

As illustrated in FIGS. 1-4, the invention is a pair of goggles 10 comprising a frame 12, a lens 14, and a pair of goggle outriggers 16: a first goggle outrigger 16a and a second goggle outrigger 16b. Typically, the pair of goggles 10 is adapted for retention upon the head of a user by the use of a strap 20.

All parts of the pair of goggles 10 can conveniently be made from a lightweight plastic material. The lens 14 is typically made from polycarbonate. The other parts of the pair of goggles 10 are typically made from nylon.

The frame 12 defines a central opening 22, and has a middle portion 24, a first side portion 26 and a second side portion 28. Each side portion has a pair of opposed goggle outrigger strut attachment devices 30. As discussed below, in the embodiment illustrated in the drawings, each goggle outrigger strut attachment device 30 is provided by a bore 46.

The lens 14 is attached to the frame 12 within the central opening 22.

The first goggle outrigger 16a is attached to the first side portion 26 of the frame 12. The first goggle outrigger 16a comprises a goggle outrigger body 32 and a pair of opposed struts 34. Each opposed strut 34 of the first goggle outrigger 16a has a proximal end 36 attached to the goggle outrigger body 32 and a free distal end 38. Each distal end 38 of each strut 34 on the first goggle outrigger 16a has a frame attachment device 40.

The second goggle outrigger 16b is attached to the second side portion 28 of the frame 12. In similar fashion to that of the first goggle outrigger 16a, the second goggle outrigger 16b comprises a goggle outrigger body 32 and a pair of opposed struts 34. Each opposed strut 34 of the second goggle outrigger 16b has a proximal end 36 attached to the goggle outrigger body 32 and a free distal end 38. Each distal end 38 of each strut 34 on the second goggle outrigger 16b also has a frame attachment device 40.

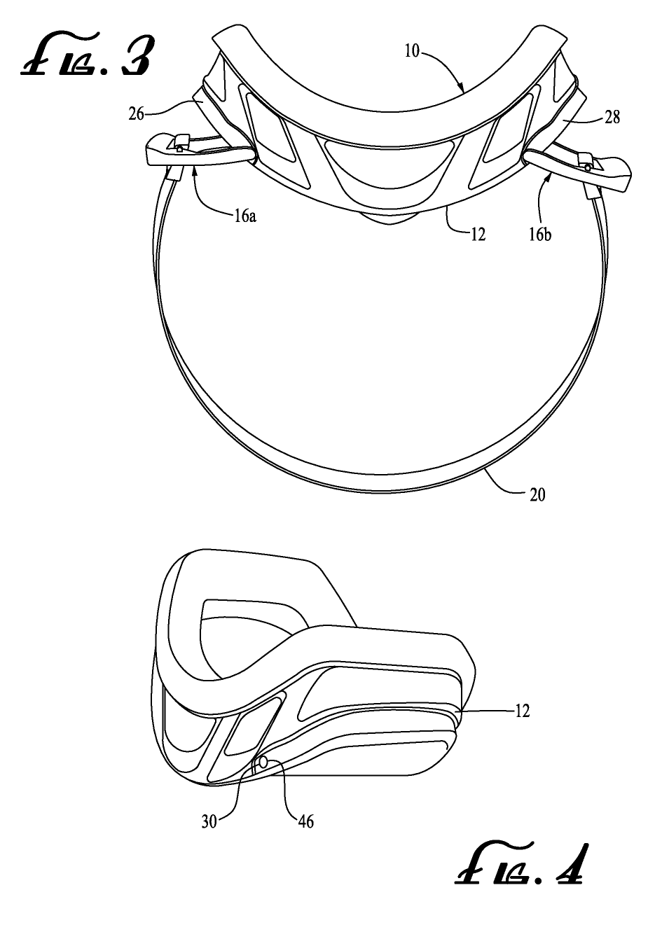

As best seen in FIG. 3, each frame attachment device 40 on each distal end 38 of each strut 34 of each goggle outrigger 16 is attached to a goggle outrigger strut attachment device 30 on the frame 12, such that each goggle outrigger 16 is hingedly affixed to the frame 12, and such that the goggle outrigger body 32 of each goggle outrigger 16 can be articulated away from the frame 12.

In the invention, each goggle outrigger 16 can be easily deattached from and reattached to the frame 12 without the use of tools. In the embodiment illustrated in the drawings, this is easily accomplished because of the following features. The distal ends 38 of both goggle outrigger struts 34 can be flexed by the fingers of a user a distance of at least about ⅛ inch, the frame attachment devices 40 at the distal end 38 of each goggle outrigger strut 34 comprises a goggle outrigger prong 44, and each goggle outrigger strut attachment device 30 is a goggle outrigger bore 46 in the frame 12 capable of accepting and retaining one of the goggle outrigger prongs 44.

As illustrated in the embodiments shown in the drawings, the pair of goggles 10 of the invention can also comprise a strap outrigger 48 attached to both the goggle outrigger body 32 of the first goggle outrigger 16a and the goggle outrigger body 32 of the second goggle outrigger 16b. Both the first and second strap outriggers 48 comprise a strap outrigger body 50 and at least one goggle outrigger body attachment device 52.

In the embodiment illustrated in the drawings, each strap outrigger 48 comprises a pair of opposed goggle outrigger body attachment devices 52, and each goggle outrigger body attachment device 52 comprises a strap outrigger prong 54 adapted to be disposed and retained by a strap outrigger bore 56 defined within the body 32 of each goggle outrigger 16. Such configuration allows each strap outrigger 48 to freely articulate away from the frame 12.

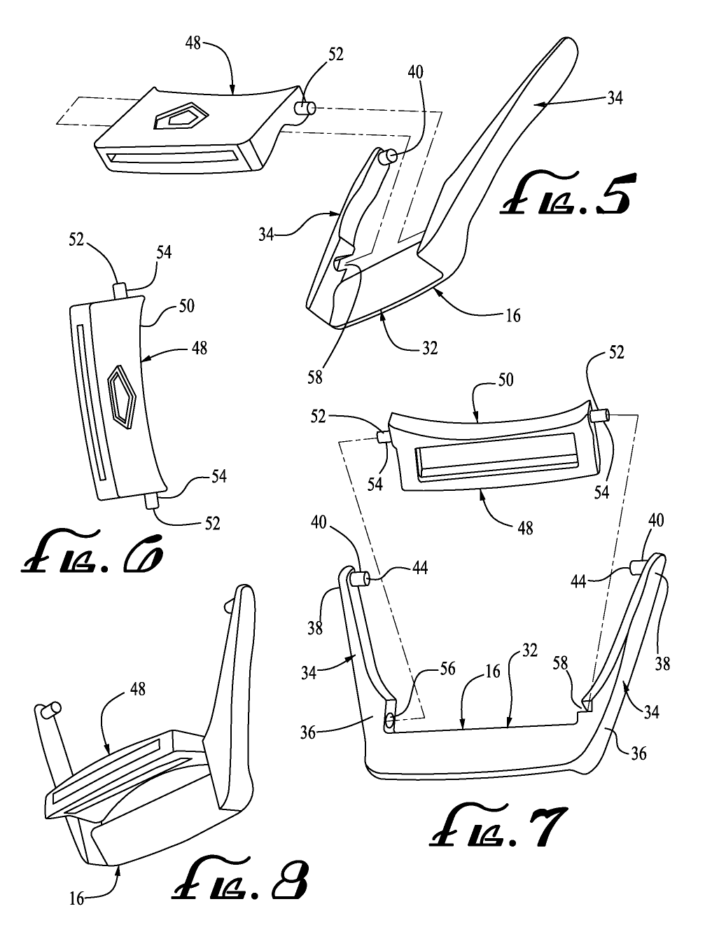

As illustrated in FIGS. 5-8, one of the pair of strap outrigger bores 56 within each strap outrigger body 50 of each goggle outrigger 16 further comprises a slot 58 to facilitate the easy attachment to and deattachment from a goggle outrigger 16 by each strap outrigger 48 without the use of tools.

The invention allows the user of the pair of goggles to periodically and conveniently replace the pair of goggle outriggers with goggle outriggers of different color and/or design.

The invention further allows the user to periodically and conveniently replace the pair of strap outriggers with strap outriggers of different color and/or design.

Although the description above contains many specifications, these should not be construed as limiting the scope of the invention but as merely providing illustrations of some of the presently preferred embodiments of this invention.

What is claimed is:

1. A pair of goggles comprising:
(a) a frame defining a central opening, the frame having a middle portion, a first side portion and a second side portion, each side portion having a pair of opposed goggle outrigger strut attachment devices;
(b) a lens attached within the central opening of the frame;
(c) a first goggle outrigger attached to the first side portion of the frame, the first goggle outrigger comprising a body and a pair of opposed struts, each opposed strut of the first goggle outrigger having a proximal end attached to the body of the first goggle outrigger and a free distal end, each distal end having a frame attachment device; and
(d) a second goggle outrigger attached to the second side portion of the frame, the second goggle outrigger comprising a body and a pair of opposed struts, each opposed strut of the second goggle outrigger having a proximal end attached to the body of the second goggle outrigger and a free distal end, each distal end having a frame attachment device;
wherein each frame attachment device on each distal end of each strut of each goggle outrigger is attached to a goggle outrigger strut attachment device on the frame, such that each goggle outrigger is hingedly affixed to the frame, and such that the body of each goggle outrigger can be articulated away from the frame; and
wherein each goggle outrigger can be easily deattached from and reattached to the frame without the use of tools.

2. The pair of goggles of claim 1 wherein the distal ends of both goggle outriggers can be flexed by the fingers of the user a distance of at least about ⅛ inch and wherein the frame attachment device at the distal end of each goggle outrigger comprises a prong.

3. A pair of goggles comprising:
(a) a frame defining a central opening, the frame having a middle portion, a first side portion and a second side portion, each side portion having a pair of opposed goggle outrigger strut attachment devices;
(b) a lens attached within the central opening of the frame;
(c) a first goggle outrigger attached to the first side portion of the frame, the first goggle outrigger comprising a body and a pair of opposed struts, the body of the first goggle outrigger having at least one strap outrigger attachment device, each opposed strut of the first goggle outrigger having a proximal end attached to the body of the first goggle outrigger and a free distal end, each distal end having a frame attachment device;
(d) a second goggle outrigger attached to the second side portion of the frame, the second goggle outrigger comprising a body and a pair of opposed struts, the body of the second goggle outrigger having at least one strap outrigger attachment device, each opposed strut of the second goggle outrigger having a proximal end attached to the body of the second goggle outrigger and a free distal end, each distal end having a frame attachment device;
(e) a first strap outrigger attached to the body of the first goggle outrigger, the first strap outrigger comprising a body and at least one goggle outrigger body attachment device; and
(f) a second strap outrigger attached to the body of the second goggle outrigger, the second strap outrigger comprising a body and at least one goggle outrigger body attachment device;
wherein each frame attachment device on each distal end of each strut of each goggle outrigger is attached to a goggle outrigger strut attachment device on the frame, such that each goggle outrigger is hingedly affixed to the frame, and such that the body of each goggle outrigger can be articulated away from the frame; and
wherein each goggle outrigger body attachment device is attached to a strap outrigger attachment device such that each strap outrigger can easily be deattached from and reattached to a goggle outrigger without the use of tools.

4. The pair of goggles of claim 3 wherein each strap outrigger is attached to the body such that it can freely articulate away from the body.

5. The pair of goggles of claim 3 wherein each strap outrigger body comprises a slot to facilitate the easy attachment to and deattachment from a goggle outrigger by each strap outrigger without the use of tools.

6. The pair of goggles of claim 3 wherein each outrigger body attachment device comprises a prong.

7. A pair of goggles comprising:
(a) a frame defining a central opening, the frame having a middle portion, a first side portion and a second side portion, each side portion having a pair of opposed goggle outrigger strut attachment devices;
(b) a lens attached within the central opening of the frame;
(c) a first goggle outrigger attached to the first side portion of the frame, the first goggle outrigger comprising a body and a pair of opposed struts, the body of the first goggle outrigger having at least one strap outrigger attachment device, each opposed strut of the first goggle outrigger having a proximal end attached to the body of the first goggle outrigger and a free distal end, each distal end having a frame attachment device;
(d) a second goggle outrigger attached to the second side portion of the frame, the second goggle outrigger comprising a body and a pair of opposed struts, the body of the second goggle outrigger having at least one strap outrigger attachment device, each opposed strut of the second goggle outrigger having a proximal end attached to the body of the second goggle outrigger and a free distal end, each distal end having a frame attachment device;
(e) a first strap outrigger attached to the body of the first goggle outrigger, the first strap outrigger comprising a body and at least one goggle outrigger body attachment device; and
(f) a second strap outrigger attached to the body of the second goggle outrigger, the second strap outrigger comprising a body and at least one goggle outrigger body attachment device;
wherein each frame attachment device on each distal end of each strut of each goggle outrigger is attached to a goggle outrigger strut attachment device on the frame, such that each goggle outrigger is hingedly affixed to the frame, and such that the body of each goggle outrigger can be articulated away from the frame;
wherein each goggle outrigger can be easily deattached from and reattached to the frame without the use of tools; and
wherein each goggle outrigger body attachment device is attached to a strap outrigger attachment device such that each strap outrigger can easily be deattached from and reattached to a goggle outrigger without the use of tools.

8. The pair of goggles of claim 7 wherein each goggle outrigger body attachment device comprises a prong.

9. The pair of goggles of claim 7 wherein the distal ends of both goggle outriggers can be flexed by the fingers of the user a distance of at least about ⅛ inch and wherein the frame attachment device at the distal end of each goggle outrigger comprises a prong.

10. The pair of goggles of claim 7 wherein each strap outrigger is attached to the body such that it can freely articulate away from the body.

11. The pair of goggles of claim 7 wherein each strap outrigger body comprises a slot to facilitate the easy attachment to and deattachment from a goggle outrigger by each strap outrigger without the use of tools.
